(19) United States
(12) Patent Application Publication
(10) Pub. No.: US 2011/0171070 A1
(43) Pub. Date: Jul. 14, 2011

(54) SURFACE-MODIFIED SENSOR DEVICE AND METHOD FOR SURFACE-MODIFYING THE SAME

(75) Inventors: Shu-Ting CHANG, Taipei City (TW); Hsiao-Ling Yeh, New Taipei City (TW); Yu-Chia Tsao, Taipei City (TW); Jung-Chien Chang, New Taipei City (TW); Jia-Huey Tsao, New Taipei City (TW); Ko-Shao Chen, Taipei City (TW)

(73) Assignee: Forward Electronics Co., Ltd., Taipei City (TW)

(21) Appl. No.: 13/069,798

(22) Filed: Mar. 23, 2011

Related U.S. Application Data
(63) Continuation-in-part of application No. 12/153,911, filed on May 28, 2008.

(30) Foreign Application Priority Data
Jan. 13, 2011 (TW) 100101243

(51) Int. Cl. G01N 33/48; H05H 1/24; C08F 2/48

(52) U.S. Cl. 422/68.1; 427/569; 427/508

(57) ABSTRACT

A method for surface-modifying a sensor device is disclosed, which includes the following steps: providing a sensor device, wherein a surface of the sensor device has a metal film; forming a surface-modification layer having a plurality of carboxyl groups on the metal film of the sensor device by isopropyl alcohol plasma; and forming a poly(acrylic acid) layer on the surface-modification layer, wherein the acrylic acid of the poly(acrylic acid) layer is grafted to the carboxyl of the surface-modification layer. A surface-modified sensor device is also disclosed.

This patent application is a continuation-in-part (CIP) of U.S. patent application Ser. No. 12/153,911, filed May 28, 2008, entitled “Method for Biomolecule Immobilization”, herein incorporated by reference.

### BACKGROUND OF THE INVENTION

1. Field of the Invention

The present invention relates to a surface-modified sensor device and a method for surface-modifying a sensor device and, more particularly, to a surface-modified sensor device with increased density of bonded molecules and uniformity and a method for surface-modifying a sensor device.

2. Description of Related Art

In recent years, the application of optical sensors has become a major trend in biomolecule detection for medical diagnosis and film thickness measurement. In the biomolecule detection, the biomolecules are required to be immobilized on the sensor devices and then reacted with the test sample to provide a signal variation for the determination of the species and amount of the test sample.

If the biomolecules require to be immobilized on the metal coatings of the optical sensors, the metal coatings need to be modified first. In the conventional surface modification for the biomolecules, the optical sensors are immersed in an 11-mercaptoundecanoic acid (MUA) solution. By way of the immersion, it is expected that the lone pair of sulfur in MUA will occupy an outer vacant orbital of a metal atom to form a stable coordination bond therebetween. Accordingly, carboxyl groups (COOH) are formed on the metal coatings to achieve the modification thereof. Subsequently, the carboxyl groups of the surface modification layer are bonded to biomolecules in the presence of a coupling activator, 1-ethyl-3-(3-dimethylaminopropyl)carbodiimide (EDC) N-hydroxysuccinimide (NHS), to realize biomolecule immobilization.

Nevertheless, such chemical modification of immersion in MUA involves considerable reaction time and causes the metal coatings to have uneven surface hydrophilicity, leading to undesirable result of the modification. Accordingly, the modification cannot achieve the anticipated level and has drawbacks such as long waiting time, increased experimental instability, and reduced uniformity.

Therefore, it is desirable to provide a method for surface-modifying a sensor device to give the metal coating of the sensor device uniform surface hydrophilicity so that the detection properties, sensitivity, and so on of the sensor device can be improved to benefit the accuracy of the of the biomolecule detection.

### SUMMARY OF THE INVENTION

The object of the present invention is to provide a method for surface-modifying a sensor device. The method can increase the number of carboxyl groups and hydrophilicity of the surface of the sensor device, thereby enhancing the immobilization of the biomolecules.

To achieve the object, one aspect of the present invention provides a method for surface-modifying a sensor device including the following steps: providing a sensor device, wherein a surface of the sensor device has a metal film; forming a surface-modification layer having a plurality of carboxyl groups on the metal film of the sensor device by isopropyl alcohol plasma; and forming a poly(acrylic acid) layer on the surface-modification layer, wherein the acrylic acid of the poly(acrylic acid) layer is grafted to the carboxyl groups of the surface-modification layer.

In the abovementioned method of the present invention, after the treatment of isopropyl alcohol plasma, carboxyl groups (COOH) can be formed on the metal film of the sensor device and subsequently grafted with acrylic acid by polymerization so that a poly(acrylic acid) layer can be formed on the sensor device. Meanwhile, the time for performance of the plasma modification can be in a range from 1 to 30 minutes, or from 5 to 15 minutes. During the performance of the plasma modification, the strength of watts or pressure can be determined on the kind of the plasma, the time of the performance, and so on.

Compared with a sensor device modified only with isopropyl alcohol plasma, much more carboxylic groups, more uniform distribution of the carboxylic groups, and better hydrophilicity are introduced in the sensor device of the present invention treated with the combination of the isopropyl alcohol plasma modification and the acrylic acid polymerization so as to benefit subsequent immobilization of bio-molecules, leading to improvement of sensitivity and detection properties of the sensor device.

In the abovementioned method for surface-modifying a sensor device, the kind of the sensor device is not limited and it can be, for example, an optical fiber sensor device. Also, the kind of the metal film on the sensor device is not limited. However, the metal film can be a gold or silver film in order to give the optical sensor device a preferable reaction. In general, a gold film is used as the metal film. The thickness of the film is not limited and is preferably in a range from 20 nm to 80 nm, for example 40±5 nm. The formation of the film is also not limited and it can be any manner used by a person skilled in the art of the present invention, for example electroplating or arranging metal nanoballs to form a film.

Therefore, if a metal film on a sensing area of an optical fiber sensor device is treated with the method of the present invention and then bio-molecules are immobilized thereon, this device can be used to detect a sample according to surface plasmon resonance (SPR).

The abovementioned method for surface-modifying a sensor device can further include the following step: forming a bio-molecule layer on the poly(acrylic acid) layer, wherein bio-molecules of the bio-molecule layer are bonded to the carboxyl groups of poly(acrylic acid) of the poly(acrylic acid) layer.

The aforesaid bio-molecules can be antibodies, antigens, enzymes, parts of tissues, or single cells. For example, since protein A or serum albumin is able to bind to the Fc region of an antibody, an antigen can be specifically recognized by the antibody bonded to the protein A or serum albumin (serving as the bio-molecules) immobilized on the thin metal film. Hence, the optical sensors modified in the abovementioned method can specifically detect the antigen recognized by the antibody bonded to the protein A or serum albumin, and thus identify the antigen and its concentration.

In the abovementioned method for surface-modifying a sensor device of the present invention, the biomolecules of the bio-molecule layer are bonded to the poly(acrylic acid) layer in the presence of a coupling activator. The coupling activator can be selected from a group consisting of 1-ethyl-3-(3-dimethylaminopropyl)carbodiimide (EDC), N-hydroxy-succinimide (NHS), and a combination thereof.

In the abovementioned method for surface-modifying a sensor device, said poly(acrylic acid) grafted with the carboxyl groups can be formed by the polymerization of acrylic acid under UV illumination. In other words, under UV illumination, grafting polymerization of acrylic acid is carried on to make poly(acrylic acid) be grafted the carboxyl groups of the surface modification layer. Accordingly, more and uniform carboxyl groups can be formed on the sensor device.

Another object of the present invention is to provide a surface-modified sensor device to give better detection properties, sensitivity etc. so as to promote the accuracy of the biomolecule detection.

In order to achieve the object mentioned above, another aspect of the present invention provides a surface-modified sensor device including: a sensor device, on which a metal film is disposed; a surface-modification layer having a plurality of carboxyl groups on the metal film of the sensor device, wherein the surface-modification layer is formed by isopropyl alcohol plasma; and a poly(acrylic acid) layer on the surface-modification layer, wherein the acrylic acid of the poly(acrylic acid) layer is grafted to the carboxyl groups of the surface-modification layer.

The surface-modified sensor device of the present invention said above can further include: a bio-molecule layer located on the poly(acrylic acid) layer, wherein bio-molecules of the bio-molecule layer are bonded to the carboxyl groups of poly(acrylic acid) of the poly(acrylic acid) layer. Particularly, the biomolecules are not limited and they can be protein A or serum albumin. Besides, the biomolecules of the bio-molecule layer can be bonded to the poly(acrylic acid) layer in the presence of a coupling activator.

In the surface-modified sensor device of the present invention, the metal film can be a gold or silver film, and the poly(acrylic acid) layer can be formed by the polymerization of acrylic acid under UV illumination.

In conclusion, the present invention combines surface modification of isopropyl alcohol plasma and grafting polymerization of acrylic acid to promote the stability of the manufacturing and efficiently control the density of the bonding molecules. In addition, the surface modification formed by isopropyl alcohol plasma has considerable and evenly distributing carboxyl groups and low porosity, and thus exhibits good coverage and adherence to the metal film of the sensor device. Furthermore, grafting polymerization of acrylic acid is carried on to form a poly(acrylic acid) layer on the surface modification layer to introduce more carboxyl groups uniformly distributing on the sensor device. Therefore, the surface hydrophilicity of the sensor device is significantly enhanced. Also, the increase in the number of the carboxyl groups can benefit the subsequent immobilization of the biomolecules so as to improve the detection properties and accuracy of the sensor device.

Other objects, advantages, and novel features of the invention will become more apparent from the following detailed description when taken in conjunction with the accompanying drawings.

### BRIEF DESCRIPTION OF THE DRAWINGS

FIGS. 1A to 1D show a flowchart of the method for surface-modifying a sensor device in Example 1 of the present invention;

FIG. 1E show a perspective view of a sensor device in Example 2 of the present invention;

FIG. 2A is a FTIP (Fourier transform infrared spectroscopy) spectrum of the sensor device of Comparative Example 1 in Test Example of the present invention; and

FIG. 2B is a FTIP spectrum of the sensor device of Example 1 in Test Example of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example 1

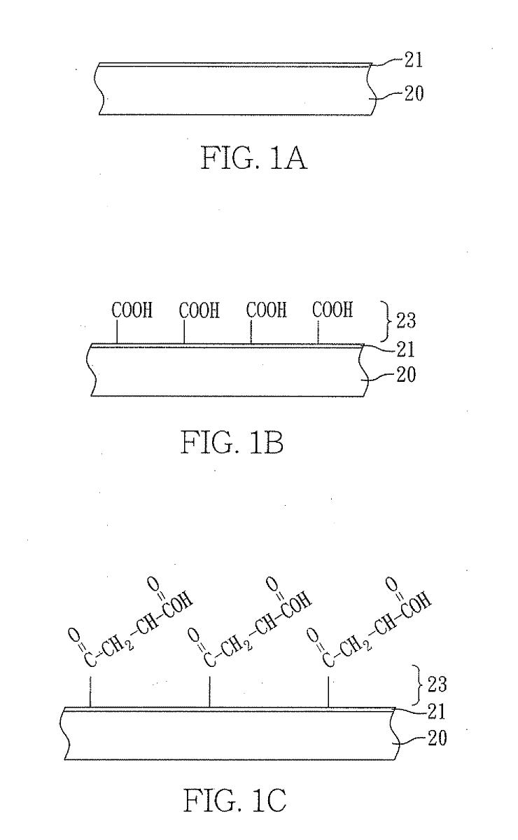

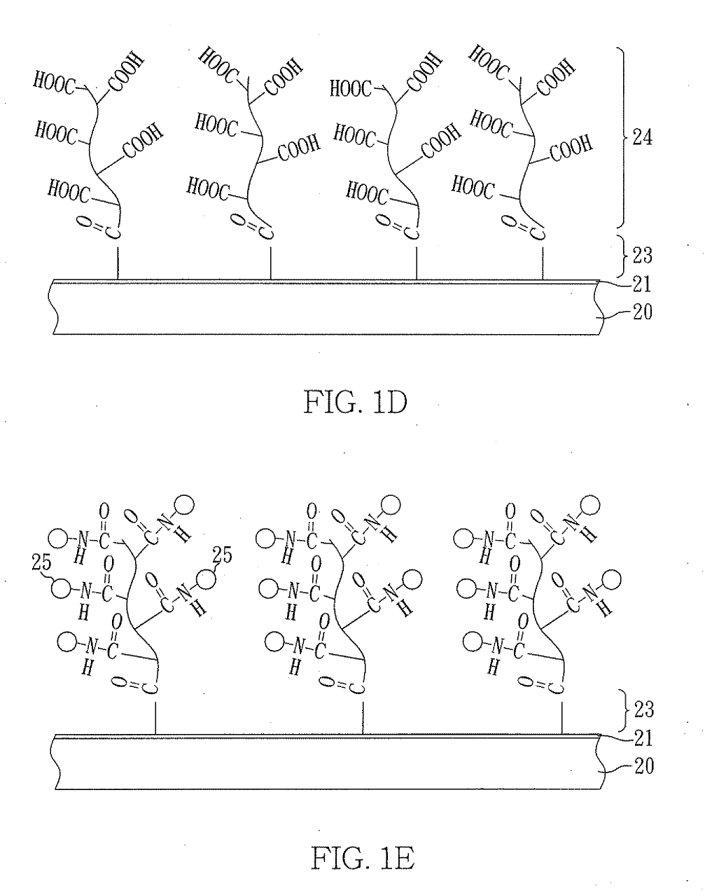

With reference to FIG. 1A to 1D, there is a flowchart of a method for surface modifying a sensor device in the present invention.

First, as shown in FIG. 1A, a sensor device 20 is provided and it has a metal film 21 disposed on a surface thereof. In the present example, the sensor device 20 is an optical fiber sensor device such as a side-polishing optical fiber sensor device, and its surface has a sensing area. On the surface of the sensing area, a gold film is formed by the method of depositing metal films such as sputtering and serves as the metal film 21.

Subsequently, as shown in FIG. 1B, a surface modification layer 23 having a plurality of carboxyl groups is formed on the metal film 21 of the sensor device 20 by isopropyl alcohol plasma. In the present example, the isopropyl alcohol plasma is carried on in the following manner. Isopropyl alcohol is used as material gas and introduced in to a vacuum discharge tube. Discharging ionizes isopropyl alcohol and then various chemical active species are produced. After complex chemical reactions, products are deposited on the metal film 21 of the sensor device 20 to form a surface modification layer 23 having a plurality of carboxyl groups. Therefore, the metal film 21 of the sensor device 20 is modified to obtain many carboxyl groups thereon. The surface modification layer 23 formed thereby has low thickness and porosity, and evenly covers the surface of the metal film 21 of the sensor device 20.

Then, as shown in FIGS. 1C and 1D, acrylic acid is used as a monomer and grafting polymerization thereof is performed under UV illumination. Hence, acrylic acid monomers are grafted to the carboxyl groups of the surface modification layer 23 and forms a poly(acrylic acid) layer 24.

### Example 2

First, as mentioned in Example 1, the surface of the sensor device 20 is modified to form the surface modification 23 and the poly(acrylic acid) layer 24 on the metal film 21 of the sensor device 20.

Subsequently, as shown in FIG. 1E, the carboxyl groups of the poly(acrylic acid) layer 24 is activated by a coupling activator. Plural biomolecules 24 are provided and their amino groups are bonded to the carboxyl groups of the poly(acrylic acid) layer 24 to form a biomolecule layer. In the present invention, 1-ethyl-3-(3-dimethylaminopropyl)carbodiimide is used as the coupling activator.

### Comparative Example 1

In the manner similar to that mentioned in Example 1, the metal film 21 of the sensor device 20 is surface-modified. However, surface modification employs only isopropyl alcohol plasma.

### Test Example

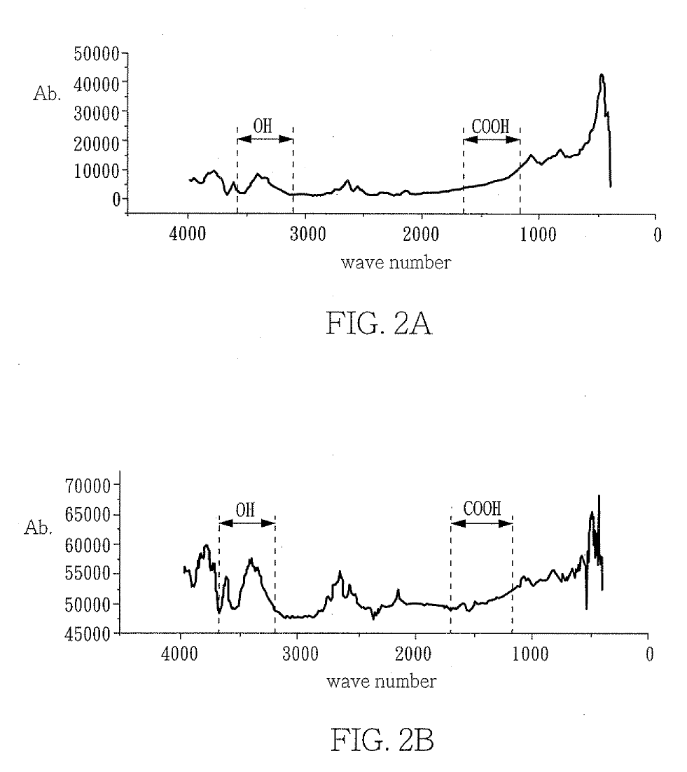

Fourier transform infrared spectroscopy analysis (FTIP analysis) is, performed to test the sensor devices made in Example 1 and Comparative Example 1, and their results are respectively shown in FIGS. 2A and 2B. FIG. 2A shows the FTIP spectrum of the sensor device of Comparative Example 1 (only surface-modified by isopropyl alcohol plasma). FIG. 2B shows the FTIP spectrum of the sensor device of Example 1 (surface-modified by isopropyl alcohol plasma and grafting polymerization of acrylic acid).

According to the FTIP spectrums, the sensor device of Example 1 (surface-modified by isopropyl alcohol plasma and grafting polymerization of acrylic acid) has more carboxyl groups and hydrophilic functional groups such as hydroxyl groups than that of Comparative Example 1 (only surface-modified by isopropyl alcohol plasma).

In conclusion, if a metal film is formed evenly by sputtering on the surface of the sensor device, SPR response can occur thereon. When a surface modification layer having carboxyl groups is formed on the metal film by isopropyl alcohol plasma, and acrylic acid used as a monomer is grafted to the carboxyl groups under UV illumination and then polymerized to form a poly(acrylic acid) layer, the functional groups of the metal film of the sensor device can be modified by the abovementioned mixture of the chemical films of the present invention. The increase on the number of the carboxyl groups on the surface can enhance the subsequent immobilization of the biomolecules and also hydrophilicity of the surface of the sensor device. Furthermore, since the immobilization of the biomolecules is enhanced, the detection efficiency can be improved and detection accuracy and reaction speed can both advanced.

Although the present invention has been explained in relation to its preferred embodiment, it is to be understood that many other possible modifications and variations can be made without departing from the scope of the invention as hereinafter claimed.

What is claimed is:

1. A method for surface-modifying a sensor device comprising the following steps:
providing a sensor device, wherein a surface of the sensor device has a metal film;
forming a surface-modification layer having a plurality of carboxyl groups on the metal film of the sensor device by isopropyl alcohol plasma; and
forming a poly(acrylic acid) layer on the surface-modification layer, wherein the acrylic acid of the poly(acrylic acid) layer is grafted to the carboxyl groups of the surface-modification layer.

2. The method as claimed in claim 1, further comprising the following step: forming a bio-molecule layer on the poly(acrylic acid) layer, wherein bio-molecules of the bin-molecule layer are bonded to the carboxyl groups of poly(acrylic acid) of the poly(acrylic acid) layer.

3. The method as claimed in claim 2, wherein the biomolecules are protein A or serum albumin.

4. The method as claimed in claim 2, wherein the biomolecules of the bio-molecule layer are bonded to the poly(acrylic acid) layer in the presence of a coupling activator.

5. The method as claimed in claim 1, wherein the metal layer is a gold or silver layer.

6. The method as claimed in claim 1, wherein the poly(acrylic acid) layer grafted to the carboxyl groups of the surface-modification layer is formed by the polymerization of acrylic acid under UV illumination.

7. A surface-modified sensor device, comprising:
a sensor device, on which a metal film is disposed;
a surface-modification layer having a plurality of carboxyl groups on the metal film of the sensor device, wherein the surface-modification layer is formed by isopropyl alcohol plasma; and
a poly(acrylic acid) layer on the surface-modification layer, wherein the acrylic acid of the poly(acrylic acid) layer is grafted to the carboxyl groups of the surface-modification layer.

8. The surface-modified sensor device as claimed in claim 7, further comprising: a bio-molecule layer located on the poly(acrylic acid) layer, wherein bio-molecules of the bio-molecule layer are bonded to the carboxyl groups of poly(acrylic acid) of the poly(acrylic acid) layer.

9. The surface-modified sensor device as claimed in claim 8, wherein the biomolecules are protein A or serum albumin.

10. The surface-modified sensor device as claimed in claim 8, wherein the biomolecules of the bio-molecule layer is bonded to the poly(acrylic acid) layer in the presence of a coupling activator.

11. The surface-modified sensor device as claimed in claim 7, wherein the metal layer is a gold or silver layer.

12. The surface-modified sensor device as claimed in claim 7, wherein the poly(acrylic acid) layer grafted to the carboxyl groups of the surface-modification layer is formed by the polymerization of acrylic acid under UV illumination.
